(19) United States
(12) Patent Application Publication
(10) Pub. No.: US 2022/0008504 A1
(43) Pub. Date: Jan. 13, 2022

(54) METHODS AND COMPOSITIONS FOR TREATMENT OF ABERRANT HEMATOPOIESIS

(71) Applicant: The Methodist Hospital, Houston, TX (US)

(72) Inventors: Longhou Fang, Katy, TX (US); Qilin Gu, Quincy, MA (US)

(73) Assignee: The Methodist Hospital, Houston, TX (US)

(21) Appl. No.: 17/297,543

(22) PCT Filed: Jan. 29, 2020
(86) PCT No.: PCT/US2020/015620

§ 371 Date: May 27, 2021

Related U.S. Application Data
(60) Provisional application No. 62/798,861, filed on Jan. 30, 2019.

(51) Int. Cl. A61K 38/17; A61K 31/047; A61K 31/4439; A61K 31/7105; A61K 31/40; A61K 31/4418; A61K 31/405; A61K 31/366; A61K 31/47; A61K 31/22; A61K 31/505; A61P 7/06

(52) U.S. Cl. CPC A61K 38/177; A61K 31/047; A61K 31/4439; A61K 31/7105; A61K 31/40; A61P 7/06; A61K 31/405; A61K 31/366; A61K 31/47; A61K 31/22; A61K 31/505; A61K 31/4418

(57) ABSTRACT

Embodiments of the present disclosure pertain to methods of reducing hematopoiesis in a subject by administering to the subject a therapeutically effective amount of an inhibitor of Srebp2. Additional embodiments of the present disclosure pertain to compositions for reducing hematopoiesis in a subject. In some embodiments, the compositions include a therapeutically effective amount of an inhibitor of Srebp2. Further embodiments of the present disclosure pertain to methods of enhancing hematopoiesis in a subject by administering to the subject a therapeutically effective amount of an active ingredient. Additional embodiments of the present disclosure pertain to compositions for enhancing hematopoiesis in a subject. In some embodiments, the compositions of the present disclosure include active ingredients that enhance hematopoiesis in the subject.

### CROSS-REFERENCE TO RELATED APPLICATIONS

This application claims priority to U.S. Provisional Patent Application No. 62/798,861, filed on Jan. 30, 2019. The entirety of the aforementioned application is incorporated herein by reference.

### STATEMENT REGARDING FEDERALLY SPONSORED RESEARCH

This invention was made with government support under Grant No. HL114734, awarded by the National Institutes of Health; and Grant No. HL132155, awarded by the National Institutes of Health. The government has certain rights in the invention.

### BACKGROUND

Treatment options for diseases and conditions associated with increased or decreased numbers of blood cells remain limited. Numerous embodiments of the present disclosure address the aforementioned limitations by providing compositions and methods for reducing or enhancing hematopoiesis in subjects.

### SUMMARY

In some embodiments, the present disclosure pertains to methods of reducing hematopoiesis in a subject by administering to the subject a therapeutically effective amount of an inhibitor of sterol regulatory element-binding protein 2 (Srebp2). Additional embodiments of the present disclosure pertain to compositions for reducing hematopoiesis in a subject. In some embodiments, the compositions of the present disclosure include a therapeutically effective amount of an inhibitor of Srebp2.

In some embodiments, the Srebp2 inhibitors include, without limitation, small molecule inhibitors of Srebp2, Srebp2 antagonists, inhibitors of Srebp2 gene expression, and combinations thereof. In some embodiments, the Srebp2 inhibitor is a small molecule inhibitor of Srebp2, such as betulin and fatostatin.

In some embodiments, the Srebp2 inhibitors and compositions of the present disclosure may be administered to subjects that are suffering from a condition associated with increased numbers of blood cells. As such, in some embodiments, the methods of the present disclosure may be utilized to treat the condition associated with increased numbers of blood cells in the subject. In some embodiments, the condition includes, without limitation, leukemia, multiple myeloma, thrombocytosis, hyperlipidemia, hypercholesterolemia, and combinations thereof.

In additional embodiments, the present disclosure pertains to methods of enhancing hematopoiesis in a subject by administering to the subject a therapeutically effective amount of an active ingredient. Additional embodiments of the present disclosure pertain to compositions for enhancing hematopoiesis in a subject. In some embodiments, the compositions of the present disclosure include active ingredients that enhance hematopoiesis in the subject.

In some embodiments, the active ingredients of the present disclosure include, without limitation, Srebp2; an active fragment of Srebp2; a Srebp2 derivative; a nucleotide expressing Srebp2; a nucleotide expressing a Srebp2 derivative; an active fragment of a Srebp2 derivative; an activator of Srebp2 expression; Srebp2 agonists; lipids, carbohydrates or small molecules having Srebp2 agonist activity; and combinations thereof. In some embodiments, the active ingredient is Srebp2.

In some embodiments, the active ingredients and compositions of the present disclosure may be administered to subjects that are suffering from a condition associated with decreased numbers of blood cells. As such, in some embodiments, the methods of the present disclosure may be utilized to treat the condition associated with decreased numbers of blood cells in the subject. In some embodiments, the condition includes, without limitation, anemia, cytopenia, sickle cell disease, leukemia, lymphoma, and combinations thereof.

### DESCRIPTION OF THE DRAWINGS

FIG. 1A illustrates a method of reducing hematopoiesis in a subject.

FIG. 1B illustrates a method of enhancing hematopoiesis in a subject.

FIG. 2 illustrates the effect of Aibp2 and cholesterol on hematopoietic stem cell (HSC) emergence. FIG. 2A shows a whole-mount in situ hybridization (WISH) analysis of runx1, cmyb, rag1, and efnb2a expression. FIG. 2B shows HSC emergence in control or Aibp2-deficient cmyb.GFP; kdrl.mCherry zebrafish. FIG. 2C shows WISH analysis of runx1 in animals with the indicated treatments. Ethanol: EtOH. Arrowheads in FIG. 2A indicate dorsal aorta (DA) or thymus, and in FIG. 2B show cmyb⁺kdrl⁺ HSCs. Scale bar, 100 μm.

FIG. 3 shows the effect of Aibp2 on Srebp2 activity in endothelial cells (ECs). FIG. 3A shows DNA constructs used to make the transgenic zebrafish with heat shock-induced Aibp2 expression. SS: secretion signal. FIGS. 3B and 3C show qRT-PCR analyses of srebf1 and srebf2 in the AGM regions of Aibp2 overexpression (FIG. 3B) or knockdown zebrafish (FIG. 3C). FIG. 3D shows immunoblots of Srebp2 in HUVECs incubated with or without AIBP/HDL₃ (μg/ml) for 4 hours. **, p<0.01. P: Srebp2 precursor; N: nuclear Srebp2.

FIG. 4 shows the effect of Srebp2 on HSC emergence. FIG. 4A shows a WISH analysis of the indicated genes in WT or srebf2^{-/-} zebrafish. FIGS. 4B and 4C show WISH analyses of runx1 expression in the DA. nSrebp2 OE: nuclear Srebp2 overexpression.

FIG. 5 shows the effect of AIBP-regulated Srebp2 activity on Notch signaling. FIGS. 5A and 5B show WISH analysis of notch1b and runx1. The numerator indicates number of zebrafish with the representative phenotype, and denominator indicates the total number of animals assessed. FIG. 5C shows Srebp2 binding motif enrichment in differentially expressed gene groups. TSS: translation start site. FIG. 5D shows immunoblotting of SREBP2 and NOTCH1 in the HSPCs isolated from low LDL-C (1.826±0.089 mM; n=5) and high LDL-C (4.796±0.454 mM; n=5) subjects. LAMIN A/C serves as the loading control. FIG. 5E shows a working model. Bilateral cholesterol transport occurs between the ER and plasma membrane. AIBP-accelerated cholesterol efflux to HDL or hypercholesterolemia activates Srebp2, which transactivates Notch for hematopoiesis.

FIG. 6 shows hypercholesterolemia effect on HSPC expansion. FIG. 6A shows a FACS analysis of Lin-Sca-1⁺c-Kit⁺ HSPCs. Eight week-old male LDLR knockout mice were fed control diet, Western diet (WD), or WD in combination with betulin (600 mg/kg food) for 16 weeks, and bone marrow were isolated from these hypercholesterolemic mice for FACS analysis of Lin-Sca-1⁺c-Kit⁺ HSPCs. FIG. 6B shows quantitative FACS data of HSPCs in FIG. 6A. FIG. 6C shows the total plasma cholesterol (TC) measurements. n=10 per group. FIG. 6D shows the correlation of CD34⁺CD45⁺HSPC frequency with LDL-C levels in normal volunteers. Mean±SE; ****p<0.0001.

FIG. 7 shows that Srebp2 regulates HSPC emergence. FIG. 7A shows WISH analysis of the indicated gene expression in Srebp2-deficient or control embryos. Arrowheads indicate WISH signal in the thymus (rag1⁺) or DA (runx1⁺/cmyb⁺/efnb2a⁺). FIG. 7B shows the quantification of the WISH analysis.

FIG. 8 shows that Srebp2 augments HSPC emergence in wild type animals. FIG. 8A shows 4OHT induced expression of nSrebp2, with mCerulean3 serving as a fluorescent marker. FIG. 8B shows Western blot analysis of nSrebp2 expression using Flag antibody. FIG. 8C shows WISH analysis of the indicated gene expression in Srebp2-deficient or control embryos. Arrowheads indicate WISH signal in dorsal aorta (runx1⁺). FIG. 8D shows the quantification of runx1⁺ HSCs in the dorsal aorta.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Numerous diseases and conditions are associated with increased or decreased numbers of blood cells. For instance, conditions associated with decreased numbers of blood cells include anemia, cytopenia, sickle cell disease, leukemia, and lymphoma. Likewise, conditions associated with increased numbers of blood cells include leukemia, multiple myeloma, thrombocytosis, hyperlipidemia, and hypercholesterolemia. Treatment options for the aforementioned conditions remain limited. As such, a need exists for more effective methods for treating conditions associated with increased or decreased numbers of blood cells. Numerous embodiments of the present disclosure address the aforementioned need by providing compositions and methods for reducing or enhancing hematopoiesis in subjects.

Compositions and Methods for Reducing Hematopoiesis in a Subject

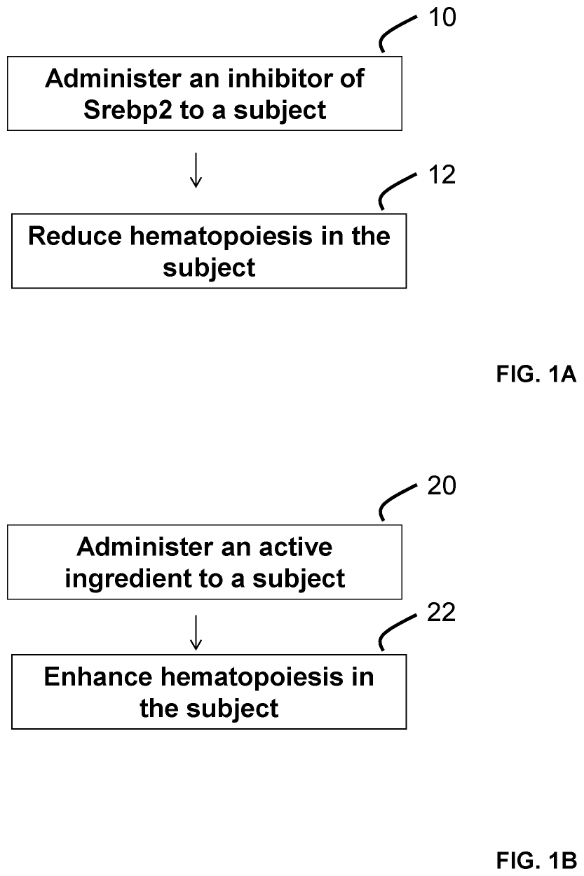

In some embodiments, the present disclosure pertains to methods of reducing hematopoiesis in a subject. In some embodiments illustrated in FIG. 1A, the methods of the present disclosure include administering to the subject a therapeutically effective amount of an inhibitor of sterol regulatory element-binding protein 2 (Srebp2) (step 10) in order to reduce hematopoiesis in the subject (step 12).

Additional embodiments of the present disclosure pertain to compositions for reducing hematopoiesis in a subject. In some embodiments, the compositions of the present disclosure include a therapeutically effective amount of an inhibitor of Srebp2.

As set forth in more detail herein, the methods and compositions of the present disclosure can have numerous embodiments. In particular, various methods may be utilized to administer various Srebp2 inhibitor compositions to various subjects. Moreover, Srebp2 inhibitors may reduce hematopoiesis in various manners.

Srebp2 Inhibitors

The methods and compositions of the present disclosure can utilize various types of Srebp2 inhibitors. For instance, in some embodiments, the Srebp2 inhibitors include, without limitation, small molecule inhibitors of Srebp2, Srebp2 antagonists, inhibitors of Srebp2 gene expression, and combinations thereof.

In some embodiments, the Srebp2 inhibitor is a small molecule inhibitor of Srebp2. In some embodiments, the small molecule inhibitor is betulin, a betulin derivative, or combinations thereof. In some embodiments, the Srebp2 inhibitor is betulin.

In some embodiments, the small molecule inhibitor is fatostatin, a fatostatin derivative, or combinations thereof. In some embodiments, the Srebp2 inhibitor is fatostatin.

In some embodiments, the Srebp2 inhibitor is an inhibitor of Srebp2 gene expression. In some embodiments, the inhibitor of Srebp2 gene expression includes an siRNA.

The Srebp2 inhibitors of the present disclosure may be in various forms. For instance, in some embodiments, the Srebp2 inhibitors are in forms that include, without limitation, nucleotides, lipids, carbohydrates (e.g., carbohydrates that over-produce a protein or augment its function), recombinant proteins, isolated proteins, protein fragments, small molecules, and combinations thereof.

Compositions for Reducing Hematopoiesis

In general, compositions for reducing hematopoiesis include one or more of the Srebp2 inhibitors of the present disclosure. In addition to Srebp2 inhibitors, the compositions of the present disclosure can include additional components.

In some embodiments, the compositions of the present disclosure also include one or more active agent stabilizers. Active agent stabilizers generally refer to compounds that are capable of reducing or preventing the degradation of the Srebp2 inhibitors of the present disclosure.

In some embodiments, the active agent stabilizers of the present disclosure include, without limitation, anti-oxidants, sequestrants, ultraviolet stabilizers, and combinations thereof.

In some embodiments, the active agent stabilizers of the present disclosure include anti-oxidants. In some embodiments, the anti-oxidants include, without limitation, vitamin E, vitamin C, triglyceride, lipids, cellulose, fibers, uric acid, glutathione, and combinations thereof. In some embodiments, the active agent stabilizers of the present disclosure include vitamin E.

In some embodiments, the active agent stabilizers of the present disclosure include sequestrants. In some embodiments, the sequestrants include, without limitation, calcium chloride, calcium acetate, calcium disodium ethylene diamine tetra-acetate, glucono delta-lactone, sodium gluconate, potassium gluconate, sodium tripolyphosphate, sodium hexametaphosphate, ethylenediaminetetraacetic acid (EDTA), and combinations thereof.

In some embodiments, the active agent stabilizers of the present disclosure include ultraviolet stabilizers. In some embodiments, the ultraviolet stabilizers include benzophenones.

The active agent stabilizers of the present disclosure may be associated with the Srebp2 inhibitors of the present disclosure in various manners. For instance, in some embodiments, the active agent stabilizers of the present disclosure may be co-encapsulated with the Srebp2 inhibitors of the present disclosure. In some embodiments, the active agent stabilizers of the present disclosure may be non-covalently associated with the Srebp2 inhibitors of the present disclosure. In some embodiments, the Srebp2 inhibitors of the present disclosure may be held in place by active agent stabilizers of the present disclosure within a composition core.

The Srebp2 inhibitors of the present disclosure may also be associated with various delivery agents. For instance, in some embodiments, the Srebp2 inhibitors of the present disclosure are associated with nanoparticles. In some embodiments, the nanoparticles have diameters ranging from about 1 nm to about 1000 nm. In some embodiments, the nanoparticles have diameters of about 50 nm to about 500 nm. In some embodiments, the nanoparticles have diameters of about 100 nm to about 150 nm. In some embodiments, the nanoparticles have diameters of about 1 nm to about 100 nm. In some embodiments, the nanoparticles have diameters of about 20 nm to about 200 nm. In some embodiments, the nanoparticles have a diameter of about 100 nm.

The nanoparticles of the present disclosure may have also various shapes. For instance, in some embodiments, the nanoparticles of the present disclosure have a spherical shape. In some embodiments, the nanoparticles of the present disclosure have a cylindrical shape. In some embodiments, the nanoparticles of the present disclosure have a circular shape. In some embodiments, the nanoparticles of the present disclosure have an elliptical shape.

Administration to Subjects

The Srebp2 inhibitors and compositions of the present disclosure may be administered to subjects in various manners. For instance, in some embodiments, the administration occurs by methods that include, without limitation, intravenous administration, subcutaneous administration (e.g., subcutaneous injection), transdermal administration, topical administration, intra-arterial administration, and combinations thereof. In some embodiments, the administration occurs by intravenous administration.

The Srebp2 inhibitors and compositions of the present disclosure may be administered to various subjects. For instance, in some embodiments, the subject is a human being. In some embodiments, the subject is suffering from a condition associated with increased numbers of blood cells. As such, in some embodiments, the methods of the present disclosure may be utilized to treat the condition associated with increased numbers of blood cells in the subject. In some embodiments, the condition includes, without limitation, leukemia, multiple myeloma, thrombocytosis, hypercholesterolemia, hyperlipidemia, and combinations thereof.

Reduction of Hematopoiesis

The administration of Srebp2 inhibitors and compositions of the present disclosure can reduce hematopoiesis in subjects in various manners. For instance, in some embodiments, the administering impairs hematopoietic stem cell (HSC) production in the subject. In some embodiments, the administering impairs hematopoietic stem and progenitor cell (HSPCs) production in the subject. In some embodiments, the administering impairs or ameliorates hypercholesterolemia or hyperlipidemia. In some embodiments, the administering impairs or ameliorates inflammation, such as an acute infection or low grade inflammation-induced HSPC expansion in the subject. In some embodiments, the administering can be utilized to treat or improve subjects suffering from leukemia of different origins (e.g., B-cell, T-cell, or myeloid leukemia), multiple myeloma, and thrombocytosis.

Compositions and Methods for Enhancing Hematopoiesis in a Subject

In additional embodiments, the present disclosure pertains to methods of enhancing hematopoiesis in a subject. In some embodiments illustrated in FIG. 1B, the methods of the present disclosure include administering to the subject a therapeutically effective amount of an active ingredient (step 20) in order to enhance hematopoiesis in the subject (step 22).

Additional embodiments of the present disclosure pertain to compositions for enhancing hematopoiesis in a subject. In some embodiments, the compositions of the present disclosure include active ingredients that enhance hematopoiesis in the subject.

As set forth in more detail herein, the methods and compositions of the present disclosure can have numerous embodiments. In particular, various methods may be utilized to administer various active ingredients to various subjects. Moreover, the active ingredients may enhance hematopoiesis in various manners.

Active Ingredients

The methods and compositions of the present disclosure can utilize various types of active ingredients for enhancing hematopoiesis. For instance, in some embodiments, the active ingredient includes, without limitation, Srebp2; an active fragment of Srebp2; a Srebp2 derivative; a nucleotide expressing Srebp2; a nucleotide expressing a Srebp2 derivative; an active fragment of a Srebp2 derivative; an activator of Srebp2 expression; Srebp2 agonists; lipids, carbohydrates or small molecules having Srebp2 agonist activity; and combinations thereof.

In some embodiments, the active ingredient is Srebp2. In some embodiments, the Srebp2 is in native, recombinant or isolated form.

In some embodiments, the active ingredient is a Srebp2 derivative. In some embodiments, the Srebp2 derivative is a polypeptide derivative of Srebp2. In some embodiments, the active ingredient is a nucleotide expressing Srebp2 or a Srebp2 derivative.

In some embodiments, the active ingredient is an activator of Srebp2 expression. In some embodiments, the activator of Srebp2 expression includes a statin. In some embodiments, the statin includes, without limitation, atorvastatin, cerivastatin, fluvastatin, lovastatin, mevastatin, pitavastatin, pravastatin, rosuvastatin, simvastatin, and combinations thereof. In some embodiments, the active ingredient is atorvastatin.

The active ingredients of the present disclosure may be in various forms. For instance, in some embodiments, the active ingredients are in forms that include, without limitation, nucleotides, lipids, carbohydrates (e.g., carbohydrates that over-produce a protein or augment its function), recombinant proteins, isolated proteins, protein fragments, small molecules, and combinations thereof.

Compositions for Enhancing Hematopoiesis

In general, compositions for enhancing hematopoiesis include one or more of the active ingredients of the present disclosure. In addition to active ingredients, the compositions of the present disclosure can include additional components.

In some embodiments, the compositions of the present disclosure also include one or more active agent stabilizers. Suitable active agent stabilizers were disclosed previously.

The active agent stabilizers of the present disclosure may be associated with the active ingredients of the present disclosure in various manners. For instance, in some embodiments, the active agent stabilizers of the present disclosure may be co-encapsulated with the active ingredients of the present disclosure. In some embodiments, the active agent stabilizers of the present disclosure may be non-covalently associated with the active ingredients of the present disclosure. In some embodiments, the active ingredients of the present disclosure may be held in place by active agent stabilizers of the present disclosure within a composition core.

The activators of the present disclosure may also be associated with various delivery agents. For instance, in some embodiments, the activators of the present disclosure are associated with nanoparticles. Suitable nanoparticles were also disclosed previously.

Administration to Subjects

The active ingredients and compositions of the present disclosure may be administered to subjects in various manners. For instance, in some embodiments, the administration occurs by methods that include, without limitation, intravenous administration, subcutaneous administration (e.g., subcutaneous injection), transdermal administration, topical administration, intra-arterial administration, and combinations thereof. In some embodiments, the administration occurs by intravenous administration.

The active ingredients and compositions of the present disclosure may be administered to various subjects. For instance, in some embodiments, the subject is a human being. In some embodiments, the subject is suffering from a condition associated with decreased numbers of blood cells in the subject. As such, in some embodiments, the methods of the present disclosure may be utilized to treat the condition associated with decreased numbers of blood cells in the subject. In some embodiments, the condition includes, without limitation, anemia, cytopenia, sickle cell disease, leukemia, lymphoma, and combinations thereof. In some embodiments, the condition includes anemia. In some embodiments, the condition includes cytopenia, such as cytopenia that can be treated with hematopoietic stem and progenitor cell transplantation. In some embodiments, the condition includes a condition that can benefit from hematopoietic stem and progenitor cell transplantation.

Enhancement of Hematopoiesis

The administration of the active ingredients and compositions of the present disclosure can enhance hematopoiesis in subjects in various manners. For instance, in some embodiments, the administering enhances hematopoietic stem cell (HSC) production in the subject. In some embodiments, the administering enhances hematopoietic stem and progenitor cell (HSPCs) production in the subject.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. AIBP-Mediated Cholesterol Efflux Instructs Hematopoietic Stem and Progenitor Cell Fate

Hypercholesterolemia, the driving force of atherosclerosis, accelerates the expansion and mobilization of hematopoietic stem and progenitor cells (HSPCs). The molecular determinants connecting hypercholesterolemia with hematopoiesis are unclear. In this Example, Applicants report that a somite-derived pro-hematopoietic cue, AIBP, orchestrates HSPC emergence from the hemogenic endothelium, a type of specialized endothelium manifesting hematopoietic potential. Mechanistically, AIBP-mediated cholesterol efflux activates endothelial Srebp2, the master transcription factor for cholesterol biosynthesis, which in turn transactivates Notch and promotes HSPC emergence.

Srebp2 inhibition impairs hypercholesterolemia-induced HSPC expansion. Srebp2 activation and Notch upregulation are associated with HSPC expansion in hypercholesterolemic human subjects. Genome-wide ChIP-seq, RNA-seq, and ATAC-seq indicate that Srebp2 trans-regulates Notch pathway genes required for hematopoiesis. Applicants' studies in this Example outline an AIBP-regulated Srebp2-dependent paradigm for HSPC emergence in development and HPSC expansion in atherosclerotic cardiovascular disease.

In particular, Applicants' findings in this Example indicate that HSPCs maintain hematopoietic output by generating the whole spectrum of blood cell lineages in vertebrate animals. Previous studies demonstrate that blood vessels play an essential role in HSPC specification in development. During embryogenesis, HSPCs are emerged from a rare population of endothelial cells (ECs) residing on the floor of the dorsal aorta (DA). Applicants' earlier studies show that apoA-I binding protein 2 (Aibp2, aka Yjefn3) regulates angiogenesis from the DA. Since HSCs arise from the ventral DA (1-3), Applicants investigated the role of Aibp2 in hematopoiesis.

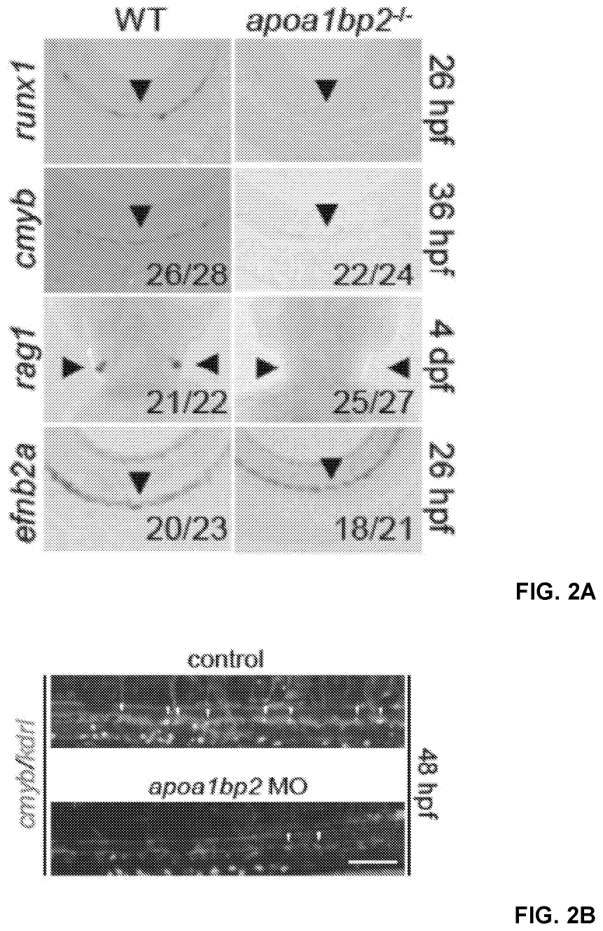

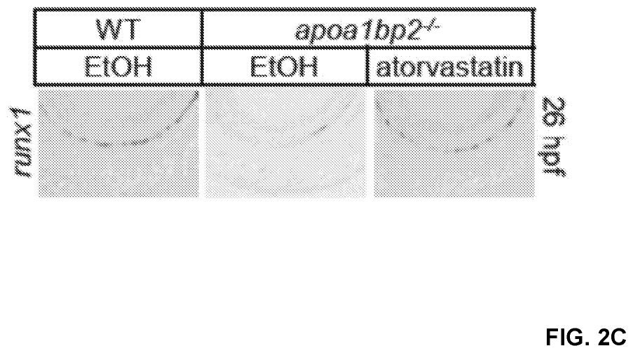

Applicants generated apoa1bp2^{-/-} zebrafish, which appeared morphologically normal. The expression of HSC marker genes runx1 and cmyb in the ventral DA, and rag1 that marks HSC-derived T-lymphocytes in the thymus, were significantly reduced in apoa1bp2^{-/-} animals (FIG. 2A). Aibp2 depletion had no observable effect on DA specification as revealed by unaffected arterial efnb2a expression (FIG. 2A). Morpholino antisense oligos (MO)-mediated Aibp2 knockdown or antibody-mediated extracellular Aibp2 neutralization reproduced Aibp2 knockout effect on hematopoiesis.

Consistently, Aibp2 deficiency reduced the number of cmyb⁺kdrl⁺ cells, which mark nascent HSCs in the ventral DA between 28 and 60 hours post-fertilization (hpf) (FIG. 2B). The results were validated using FACS analysis of cmyb⁺kdrl⁺ cells. Although blood flow regulates hematopoiesis, it appeared normal in Aibp2-deficient gata1:DsRed zebrafish. Without being bound by theory, the aforementioned data suggest that Aibp2 governs HSC ontogeny in a direct and non-cell autonomous fashion.

The expression of primitive hematopoiesis genes gata-1 and l-plastin at 24 hpf was normal in apoa1bp2 morphants whereas the marker of HSC-derived leukocytes (l-plastin⁺) at 4 days post-fertilization (dpf) was reduced. Applicants also examined the integrity of non-hematopoietic tissues by surveying the expression of associated marker genes. Development of the pronephros (cdh17), somite (desma), and sclerotome (nkx3.1) in the trunk, sonic hedgehog (shh) signaling (shha and vegfa), and arterial (dll4) and venous vasculature (ephb4) development showed no apparent changes in the absence of Aibp2. Pan-endothelial markers fli1 and kdrl were increased in Aibp2-deficient animals. These results suggest that Aibp2 plays a direct role in HSC specification.

Applicants' previous study showed increased cholesterol content in Aibp2-deficient embryos. To determine the effect of cholesterol on HSC emergence, apoa1bp2 knockouts or morphants were treated with a cholesterol-lowering drug, atorvastatin. Atorvastatin treatment restored largely runx1 expression (FIG. 2C) and reduced free cholesterol levels in Aibp2-deficient animals. Furthermore, atorvastatin expanded the cmyb⁺kdrl⁺ cells in the DA floor. These results indicate that an effective cholesterol metabolism program orchestrates HSC emergence.

Cholesterol synthesis requires the master transcription factor Srebp2, which is produced as an endoplasmic reticulum (ER)-bound precursor. Cholesterol depletion activates Srebp2 via two-step proteolytic cleavages, which releases its N-terminal transcriptional activation domain into the nucleus dictating the expression of genes for cholesterol biosynthesis such as Hmgcr, Srebf2, and cholesterol uptake Ldlr. Zebrafish genes srebf1 and srebf2 encode Srebp1 and Srebp2, respectively. Srebp1 is primarily responsible for fatty acid synthesis.

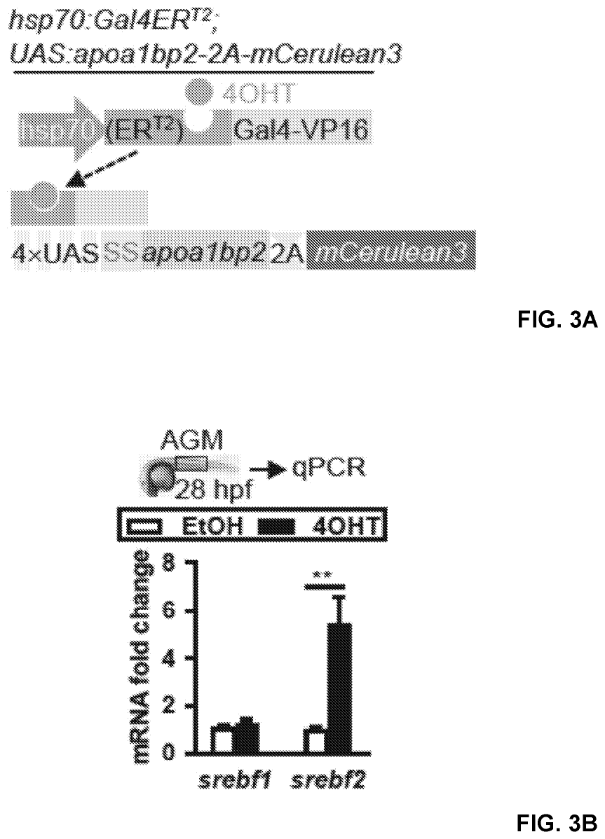

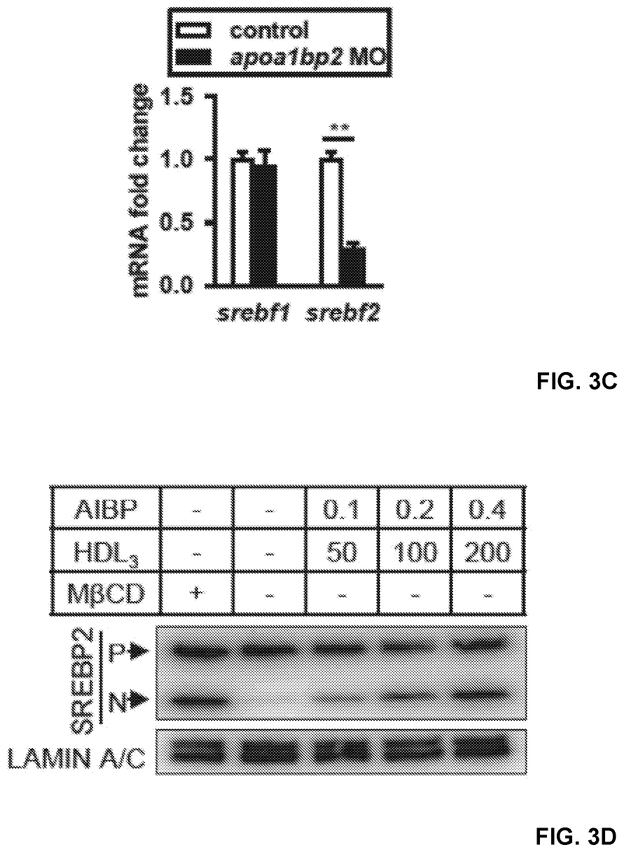

Applicants generated the double transgenic animal hsp70:Gal4ER^{T2}; UAS:apoa1bp2-2A-mCerulean3, which expressed untagged Aibp2 upon heat shock with the addition of 4-hydroxy-tamoxifen (4OHT) (FIG. 3A). Srebp2 binds its own promoter and upregulates its mRNA expression, measurement of which mirrors its transcriptional activity. Aibp2 deficiency reduced, and 4OHT-induced Aibp2 overexpression increased, srebf2 expression. However, srebf1 expression was not changed (FIGS. 3B and 3C). These results suggest that Aibp2 regulates Srebp2 activity.

Applicants also explored the hypercholesterolemia effect on embryonic hematopoiesis. High cholesterol diet (HCD)-fed adult female cmyb.GFP zebrafish produced embryos with significantly higher cholesterol content and greater srebf2 expression. The embryos produced by the HCD-fed females showed more cmyb⁺kdrl⁺ HSCs compared to the embryos produced by females fed a control diet. Similarly, hypercholesterolemic female mice produce E11.5 embryos with increased frequency of c-Kit⁺CD144⁺CD45.2⁻ and RUNX1-enriched hemogenic endothelial cells (HECs) and hematopoietic precursors. The data suggest that plasma cholesterol content regulates the developmental HSC program.

Cellular cholesterol homeostasis is sustained by LDL cholesterol uptake, Srebp2-mediated cholesterol synthesis, and HDL-mediated cholesterol efflux. Since cholesterol pools in the plasma membrane and ER are interconnected, Applicants next probed the effect of cholesterol efflux on Srebp2 activation. Cholesterol sequestrant Methyl-β-cyclodextrin (MβCD) robustly activated SREBP2 in human umbilical vein ECs (HUVECs). AIBP augments the capacity of HDL to accept cholesterol, and their combinatorial treatment dose-dependently activated SREBP2 (FIG. 3D). These results suggest that Aibp2-mediated cholesterol efflux activates SREBP2.

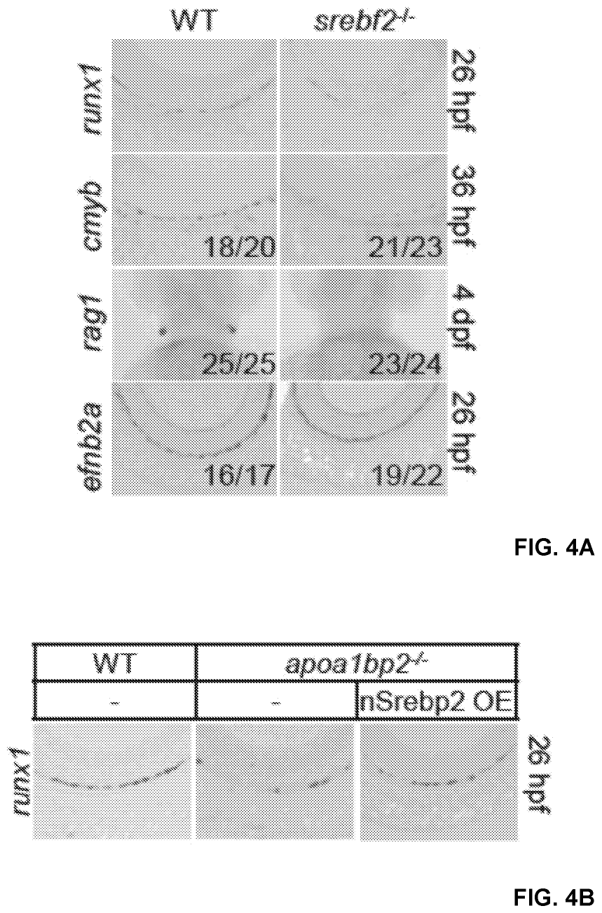

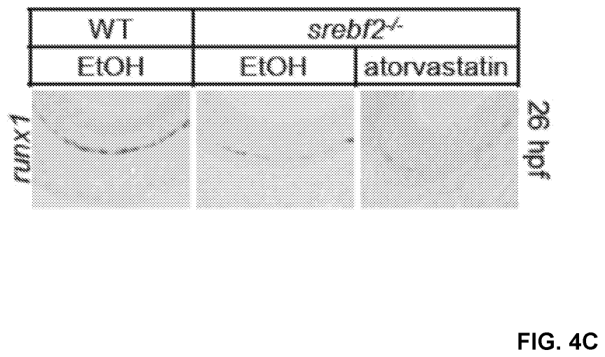

Applicants hypothesized that Srebp2 mediates Aibp2 effect on hematopoiesis. Applicants thus generated the srebf2^{-/-} zebraflsh. Srebp2 disruption markedly decreased runx1, cmyb, and rag1 expression (FIG. 4A) but did not influence efhb2a expression (FIG. 4A). Similarly, Srebp2 knockdown disrupted HSC emergence but showed no effect on DA specification, and the hematopoiesis defect was rescued by srebf2 overexpression. In addition, Srebp2 knockdown significantly reduced the cmyb⁺kdrl⁺ HSCs, but had no effect on the formation of adjacent supporting tissues, shh signaling, and arterial and venous vessel specification, while mildly increased the expression of pan-endothelial markers.

Srebp2 depletion specifically reduced the expression of Srebp2 but not Srebp1 downstream target genes. These phenotypes support Applicants' hypothesis that Aibp2, through Srebp2 activation, controls HSC emergence. To test this, Applicants created transgenic animal kdrl:Gal4ER^{T2}; UAS:Flag-nSrebp2-2A-mCerulean3, in which the addition of 4OHT induced EC-specific transcriptionally active nuclear Srebp2 (nSrebp2) expression. Nuclear srebf2 mRNA injection into Aibp2 knockouts (FIG. 4B) or 4OHT treatment of Aibp2-deficient kdrl:Gal4ER^{T2}; UAS:Flag-nSrebp2-2A-mCerulean3 animals rescued impaired HSC emergence. Atorvastatin, which activates Srebp2, markedly augmented srebf2 but not srebf1 expression. Atorvastatin treatment augmented HSC emergence, which was abolished by Srebp2 disruption (FIG. 4C). Atorvastatin-enhanced HSC emergence is not due to HSC hyper-proliferation since similar numbers of Brdu-positive cmyb⁺ cells in DA were found in control and atorvastatin-treated animals at 30 and 36 hpf. Collectively, these findings suggest that Srebp2 acts downstream of Aibp2 to orchestrate HSC specification.

The key role of Notch in HSC specification prompted Applicants to explore the role of Srebp2 in Notch signaling. Applicants employed a Notch reporter zebrafish tp1:d2GFP, which expresses an EGFP variant with shortened half-life under the control of tandem Notch responsive elements. Ablation of Aibp2 substantially reduced tp1⁺kdrl⁺ HSPCs, which can be reversed by Aibp2 overexpression. Similarly, Srebp2 depletion decreased, whereas enforced nSrebp2 expression restored tp1⁺kdrl⁺ HSPCs, in the ventral DA. Furthermore, nSrebp2 overexpression rescued HSPC emergence in apoa1bp2 morphants, indicating that Srebp2 mediates the Aibp2 effect on Notch signaling.

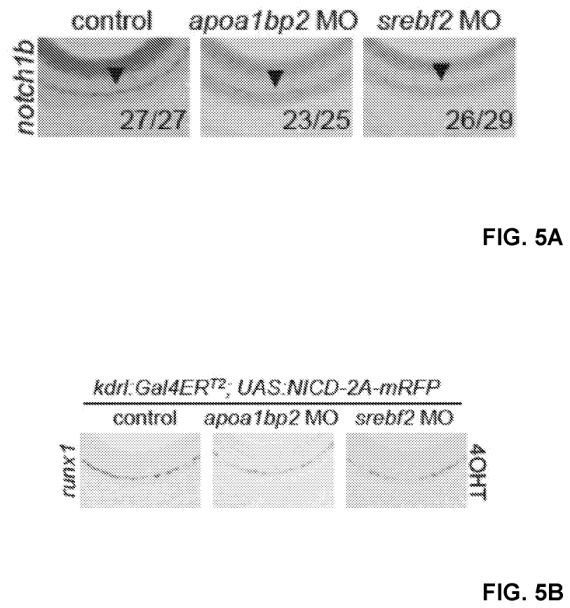

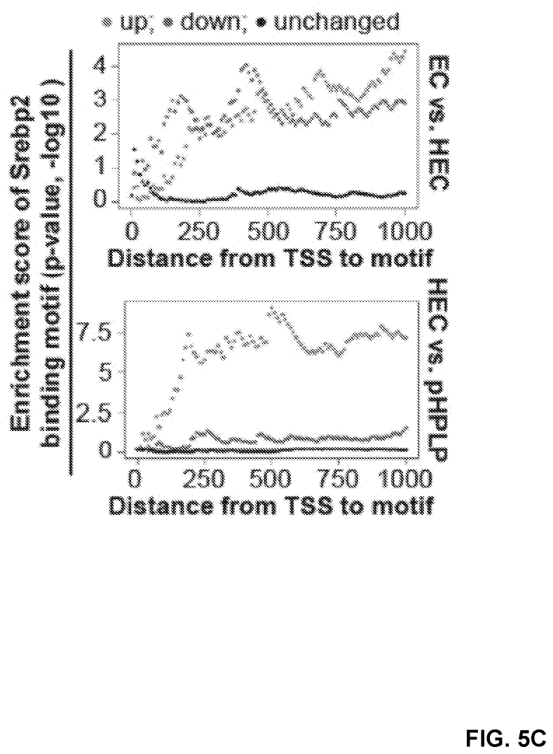

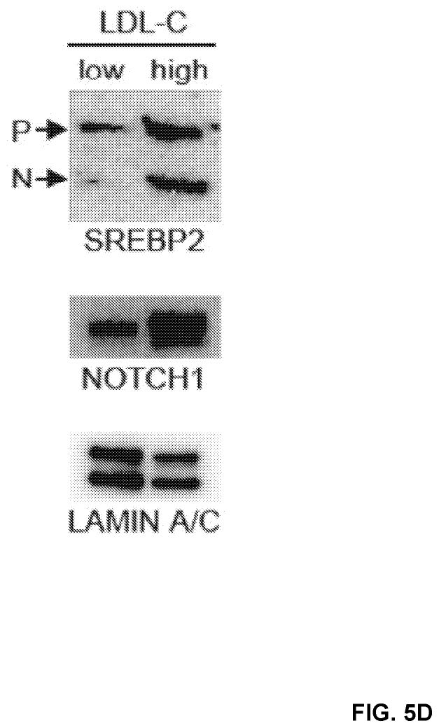

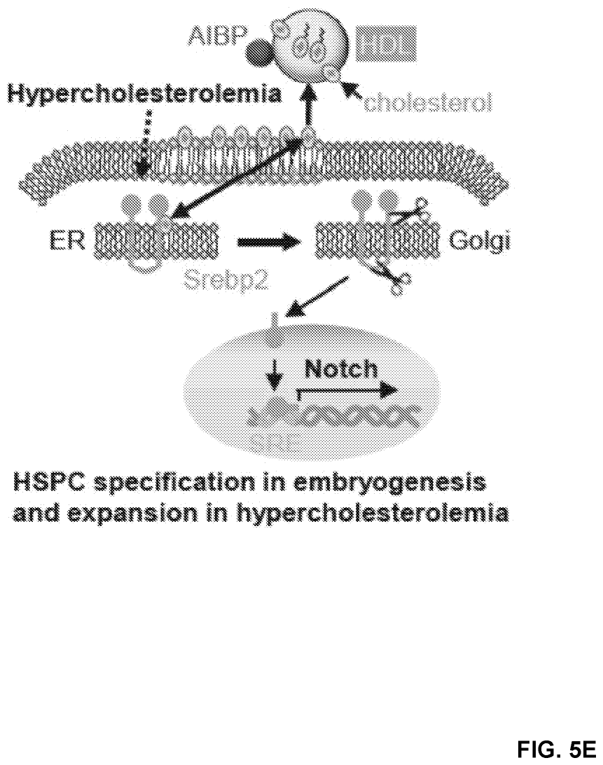

Aibp2 or Srebp2 deficiency markedly reduced the expression of notch1b but not notch1a, notch2, or notch3 in the DA (FIG. 5A), suggesting that attenuated Notch signaling caused impaired HSC emergence in apoa1bp2 or srebf2 morphants. To investigate this possibility, transgenic animals kdrl:Gal4ER^{T2}; UAS:NICD-2A-mRFP that selectively express 4OHT-inducible endothelial NICD were created. Indeed, NICD expression restored runx1 mRNA expression in apoa1bp2- or srebf2-deficient animals (FIG. 5B).

Applicants' findings agree with other findings that notch1 is intrinsically required for HSC fate. The notch1b promoter contains putative Srebp2 binding motifs, which was validated by chromatin immunoprecipitation (ChIP) and qPCR of the targeted region. By analyzing a mouse Srebp2 ChIP-seq data, Applicants found a prominent Srebp2 binding peak in the promoters of Notch1, and validated the Srebp2 binding.

Furthermore, Applicants performed a bioinformatics scan of the whole mouse genome using the putative Srebp2 binding motif, which is enriched at the center of Srebp2 ChIP-seq peaks. Applicants' results indicate that Srebp2 binding motif is highly enriched in the promoters of genes for cholesterol metabolism and Notch signaling, suggesting that this motif is highly conserved. For example, the Srebp2 binding motif and ChIP-Seq peak are present in the promoters of Srebp2-regulated cholesterol biosynthesis genes Srebf2, Hmgcr, and Ldlr, all of which were experimentally verified in murine ECs with Srebp2 overexpression. Furthermore, Srebp2 ChIP-seq results also validated Srebp2-mediated regulation of Notch signaling and cholesterol metabolism.

To further investigate the role of Srebp2 in hematopoiesis, Applicants compared gene expression profiles in paired murine ECs, a Ly6a-GFP⁺ population that contains HECs, and pre-HSCs and progenitors with lymphoid potential (pHPLPs). Compared to ECs, 752 genes are upregulated and 569 genes are downregulated in HECs, whereas compared to HECs, 752 genes are increased and 977 genes are decreased in pHPLPs. Notch pathway genes are significantly enriched in the upregulated genes of HECs compared to ECs or pHPLPs.

Except for Scap, most Srebp2-regulated cholesterol metabolism genes were repressed in HECs. SCAP is a protein chaperone of Srebp2, and is retained in the ER membrane by sterol-induced interaction with ER resident protein INSIG1/2. Scap increased up to 4-fold in HECs compared with ECs. Srebp2 binding motif or ChIP peak is markedly enriched in the promoters of upregulated genes, but to a less extent in the promoters of downregulated genes (FIG. 5C). Consistent with this, Applicants' ATAC-seq results unveiled that the Srebp2 binding motif and ChIP-seq peak are indeed located within active transcription-associated open chromatin regions of HECs, with 42% binding motifs and 79% ChIP-seq peaks overlapping the ATAC-seq peaks in HECs. Thus, Applicants' systemic bioinformatics analyses independently validate Applicants' findings that Srebp2 is a critical regulator of the Notch pathway.

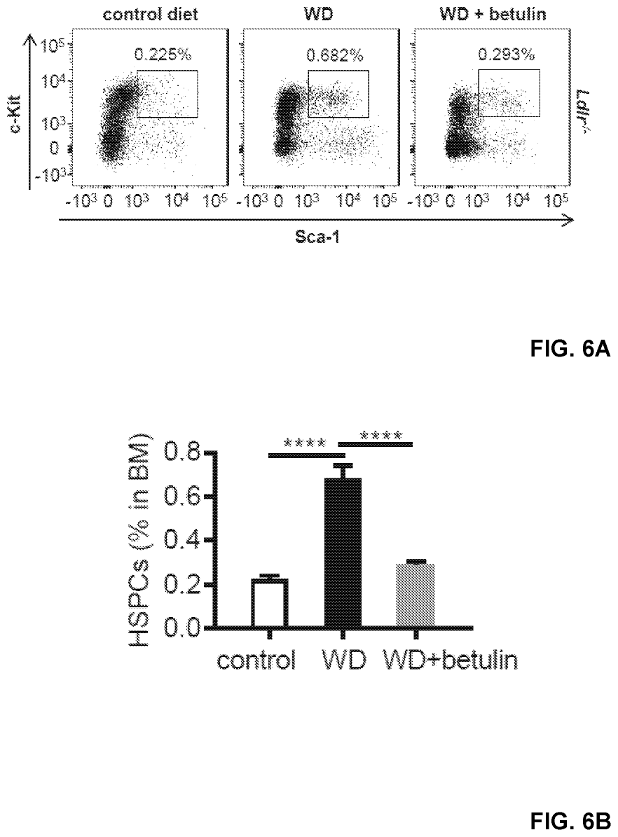

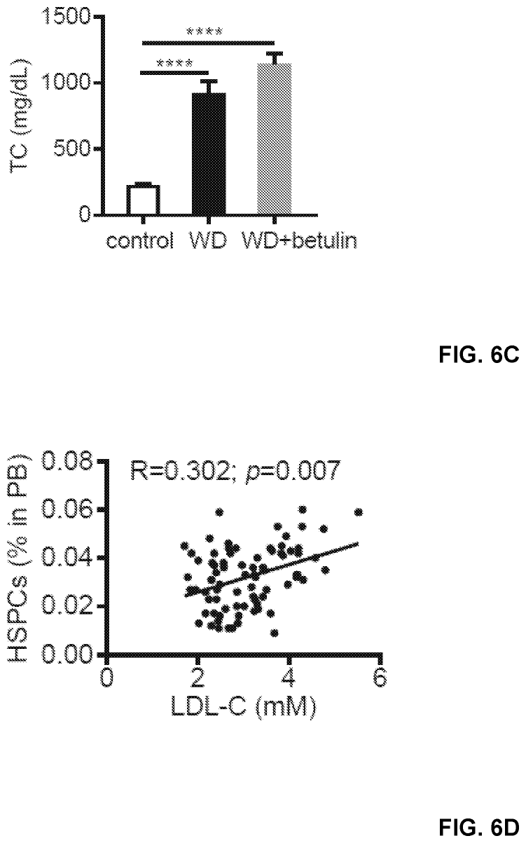

Applicants further explored the effect of hypercholesterolemia on adult hematopoiesis. As reported, Western diet (WD) feeding augmented HSPC frequency in Ldlr^{-/-} mice, and Srebp2 suppression by betulin abolished WD-induced augmentation of HSPC frequency (FIGS. 6A-C).

To relate Applicants' findings to human disease, Applicants assessed the circulating CD34⁺CD45⁺ HSPCs in healthy volunteers. Applicants found that LDL cholesterol levels are correlated with HSPC frequency, and that Srebp2 and Notch are activated/upregulated in HSPCs isolated from hypercholesterolemic subjects (FIG. 5D). Collectively, Applicants' data document a conserved Srebp2-dependent mechanism that regulates HSPC maintenance in hypercholesterolemia.

Accumulating studies indicate that Srebp2 has moonlighting activities. Applicants show that the somite-derived pro-hematopoietic Aibp2 controls hematopoiesis by targeting Srebp2-regulated cholesterol metabolism and Notch signaling (FIG. 5E). In murine HECs, only the cholesterogenic gene Scap but not others is significantly upregulated. Given that SCAP gain-of-function increases sterol-independent Srebp2 bioavailability, its upregulation may contribute to increased Srebp2 activation in HECs. Possibly, the Srebp2 function in HECs is shifted more towards Notch activation than cholesterol regulation. Applicants' findings also corroborate the essential role of somite in providing proper Notch signaling for HSC specification (e.g., Wnt16-induced Dlc/Dld presented by the sclerotome regulates Notch1b activity in the migrating HSC precursors).

Hypercholesterolemia is the driving force for atherosclerosis that underlies heart attacks and strokes. Hypercholesterolemia activates endothelial Srebp2. Srebp2 activation and Notch1 upregulation are detected in circulating HSPCs of hypercholesterolemic human subjects. Possibly, the Srebp2-regulated Notch1 signaling also orchestrates HSPC homeostasis in hypercholesterolemia. It appears that both AIBP-mediated cholesterol efflux and hypercholesterolemia converge on endothelial Srebp2 activation. Taken together, Applicants have uncovered a cholesterol metabolism pathway governing HSPC emergence in development as well as HSPC expansion in hypercholesterolemia. These insights may have relevance for hematological and cardiovascular disorders.

### Example 2, Loss of Srebp2 Impairs Hematopoiesis

This Example provides additional experimental results that demonstrate that loss of Srebp2 impairs homeostasis. As set forth in Example 1, cholesterol synthesis requires sterol responsive element binding protein 2 (Srebp2), which is encoded by the Srebf2 gene.

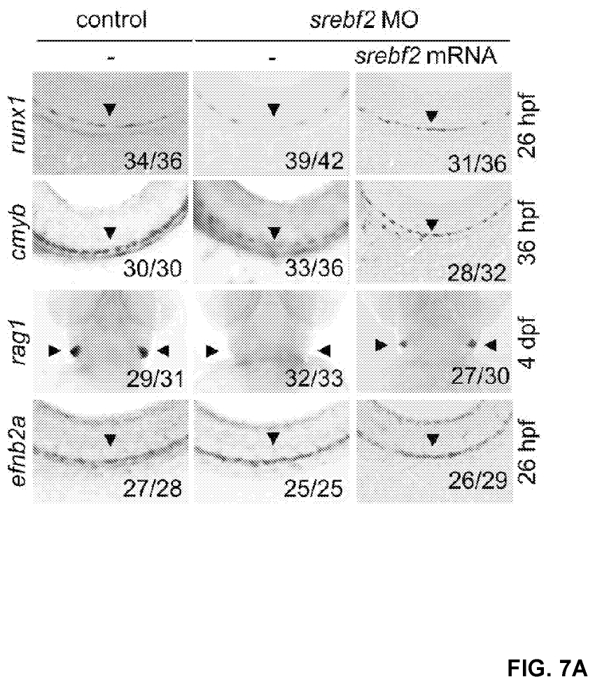

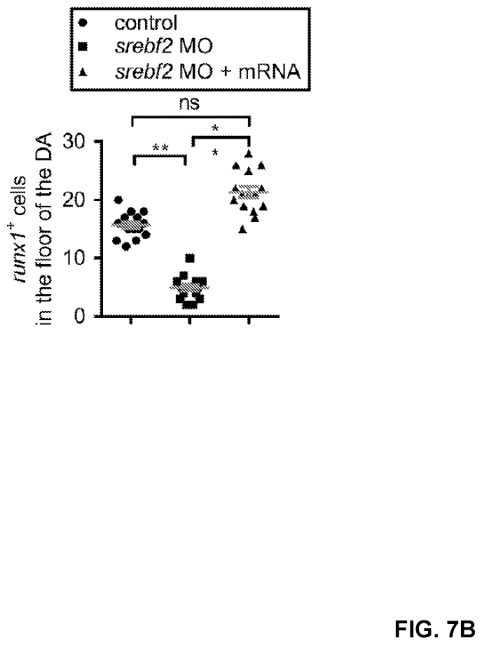

Zebrafish Srebf2 gene encodes Srebp2. The presence of HSCs in the floor of DA is defined by the expression of runx1 and cmyb. Whole-mount in situ hybridization (WISH) analyses of runx1 and cmyb showed marked decreases in their gene expression in apoa1bp2 MO compared to wild-type (WT) embryos (FIGS. 7A-7B).

T-cell lineages are believed to differentiate exclusively from HSCs, and Applicants found that T-cell development in the thymocytes was compromised as well (FIGS. 7A-7B). Given that DA is the anatomical location to generate HSCs within this time frame, Applicants also examined DA development in Aibp2-deficient animals.

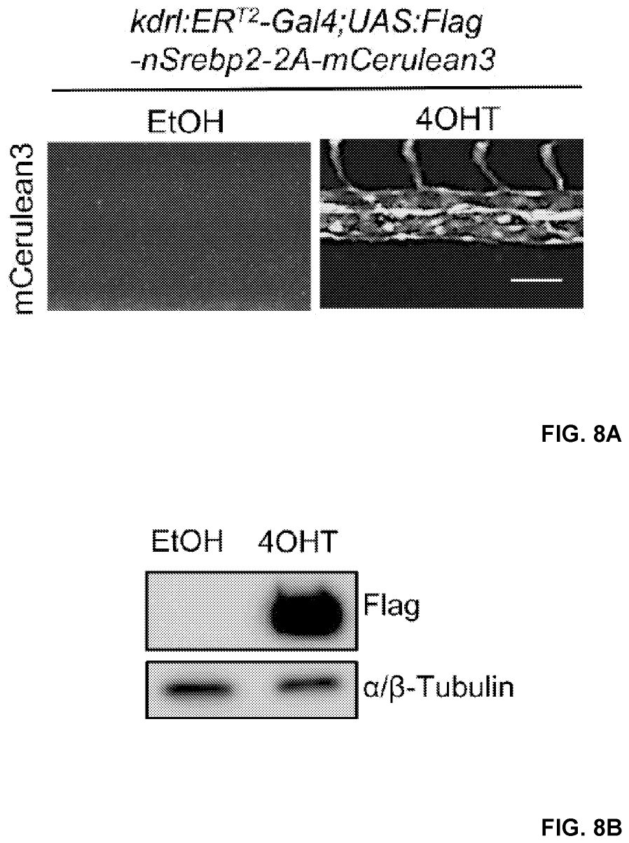

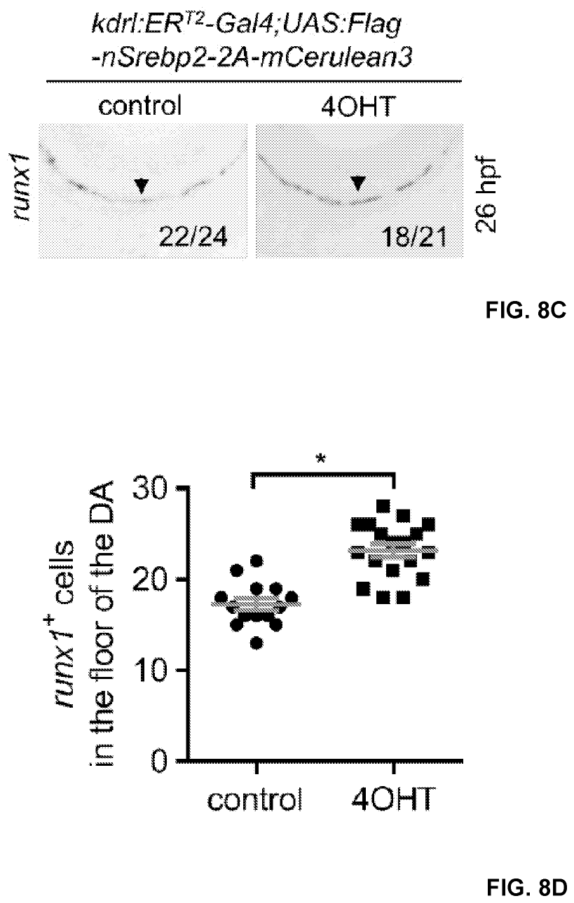

WISH analysis of efnb2a (ephrin-B2a), a DA marker, showed comparable patterns in control and Aibp2-deficient embryos (FIG. 8), suggesting that Srebp2 controls HSC specification directly. Furthermore, overexpression of Srebp2 by injecting Srebf2 mRNA rescued impaired hematopoiesis in Srebp2-deficient animals. In addition, Applicants created a transgenic animal with 4-hydroxy tamoxifen (4OHT)-induced nuclear Srebp2 (nSrebp2) expression Kdrl:ERT2-Gal4; UAS:Flag-nSrebp2-2A-mCerulean3 (FIGS. 2A-B). To test the effect of nSrebp2 overexpression on HSC emergence, 4OHT or control vehicle was added to the animals, and 4OHT-induced Srebp2 expression markedly increased HSC numbers (FIGS. 2C-D).

It has been reported that high-fed diet (HFD) augmented HSPC frequency in Ldlr^{-/-} mice. To determine the role of Srebp2 in pathological hematopoiesis, Ldlr^{-/-} mice were fed HFD to develop high low-density lipoprotein (LDL) cholesterol LDL-C levels, or with HFD with Srebp2 inhibitor betulin, and the associated effect on HSC numbers evaluated. Applicants found that botulin inhibited HFD-induced increase in HSC numbers in the bone marrow.

In summary, Applicants found that loss of Srebp2 impairs HSC production, but overexpression of Srebp2 increased HSC production (FIGS. 7-8). More specifically, Applicants discovered that Srebp2 overexpression bolsters the production of HSC (FIG. 8). Conversely, in the case of increased hematopoiesis (e.g., in leukemia or hypercholesterolemia), targeting Srebp2 using the small molecule inhibitor betulin or its derivatives may reduce HSC production.

Additional experimental results are disclosed in priority U.S. Provisional Patent Application No. 62/798,861, including Appendix B of the aforementioned provisional patent application. The entirety of the experimental results in the aforementioned provisional patent application is incorporated herein by reference.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of reducing hematopoiesis in a subject, said method comprising:
administering to the subject a therapeutically effective amount of an inhibitor of sterol regulatory element-binding protein 2 (Srebp2).

2. The method of claim 1, wherein the inhibitor of Srebp2 is selected from the group consisting of small molecule inhibitors of Srebp2, Srebp2 antagonists, inhibitors of Srebp2 gene expression, and combinations thereof.

3. The method of claim 1, wherein the inhibitor of Srebp2 is in a form selected from the group consisting of nucleotides, lipids, carbohydrates, recombinant proteins, isolated proteins, protein fragments, small molecules, and combinations thereof.

4. The method of claim 1, wherein the inhibitor of Srebp2 is a small molecule inhibitor.

5. The method of claim 4, wherein the small molecule inhibitor is betulin, a betulin derivative, fatostatin, a fatostatin derivative, or combinations thereof.

6. The method of claim 1, wherein the inhibitor of Srebp2 is an inhibitor of Srebp2 gene expression.

7. The method of claim 6, wherein the inhibitor of Srebp2 gene expression comprises an siRNA.

8. The method of claim 1, wherein the administering impairs hematopoietic stem cell (HSC) production in the subject.

9. The method of claim 1, wherein the administering impairs hematopoietic stem and progenitor cell (HSPCs) production in the subject.

10. The method of claim 1, wherein the administering impairs hypercholesterolemia-induced HSPC expansion.

11. The method of claim 1, wherein the method is utilized to treat a condition associated with increased numbers of blood cells in the subject.

12. The method of claim 11, wherein the condition is selected from the group consisting of leukemia, multiple myeloma, thrombocytosis, hyperlipidemia, hypercholesterolemia, and combinations thereof.

13. A method of enhancing hematopoiesis in a subject, said method comprising:
administering to the subject a therapeutically effective amount of an active ingredient selected from the group consisting of sterol regulatory element-binding protein 2 (Srebp2); an active fragment of Srebp2; a Srebp2 derivative; a nucleotide expressing Srebp2; a nucleotide expressing a Srebp2 derivative; an active fragment of a Srebp2 derivative; an activator of Srebp2 expression; Srebp2 agonists; lipids, carbohydrates or small molecules having Srebp2 agonist activity; and combinations thereof.

14. The method of claim 13, wherein the active ingredient is Srebp2.

15. The method of claim 14, wherein the Srebp2 is in native, recombinant or isolated form.

16. The method of claim 13, wherein the active ingredient is a Srebp2 derivative.

17. The method of claim 16, wherein the Srebp2 derivative is a polypeptide derivative of Srebp2.

18. The method of claim 13, wherein the active ingredient is a nucleotide expressing Srebp2 or a Srebp2 derivative.

19. The method of claim 13, wherein the active ingredient is an activator of Srebp2 expression.

20. The method of claim 19, wherein the activator of Srebp2 expression comprises a statin.

21. The method of claim 20, wherein the statin is selected from the group consisting of atorvastatin, cerivastatin, fluvastatin, lovastatin, mevastatin, pitavastatin, pravastatin, rosuvastatin, simvastatin, and combinations thereof.

22. The method of claim 20, wherein the statin is atorvastatin.

23. The method of claim 13, wherein the administering enhances hematopoietic stem cell (HSC) production in the subject.

24. The method of claim 13, wherein the administering enhances hematopoietic stem and progenitor cell (HSPCs) production in the subject.

25. The method of claim 13, wherein the method is utilized to treat a condition associated with decreased numbers of blood cells in the subject.

26. The method of claim 25, wherein the condition is selected from the group consisting of anemia, cytopenia, sickle cell disease, leukemia, lymphoma, and combinations thereof.
